(19) United States
(12) Patent Application Publication
(10) Pub. No.: US 2021/0046005 A1
(43) Pub. Date: Feb. 18, 2021

(54) PROCESS FOR CREATING SOLID DISPERSIONS

(71) Applicant: Mark Michael MENNING, San Rafael, CA (US)

(72) Inventor: Mark Michael MENNING, San Rafael, CA (US)

(21) Appl. No.: 16/965,498

(22) PCT Filed: Jan. 28, 2019
(86) PCT No.: PCT/US2019/015380

§ 371 Date: Jul. 28, 2020

Related U.S. Application Data
(60) Provisional application No. 62/623,127, filed on Jan. 29, 2018.

(51) Int. Cl. A61K 9/10; A61K 9/51; A61K 9/08; A61K 9/20; A61K 9/16; A61K 47/38; A61K 47/02

(52) U.S. Cl. CPC A61K 9/10; A61K 9/5115; A61K 9/08; A61K 45/06; A61K 9/16; A61K 47/38; A61K 47/02; A61K 9/20

(57) ABSTRACT

A process for producing a solid dispersion of a material by spraying the material onto a fluidized cloud of carrier particles in a fluid bed. The fluidized cloud of carrier particles is formed by adding a solid or liquid carrier to a fluid bed. The material to be sprayed is in a liquid or gas form. The process is especially applicable to the formation of amorphous solid powder pharmaceutical ingredients.

### CROSS-REFERENCE TO RELATED APPLICATION

This application claims the benefit under 35 USC 119(e) of U.S. Provisional Application No. 62/623,127, filed on Jan. 29, 2018, which is incorporated by reference in its entirety.

### BACKGROUND OF THE INVENTION

The production of solid dispersions is an important step in the manufacture of many pharmaceuticals, foodstuffs, flavorings, dietary supplements, fragrances, cosmetics, coatings, specialty chemicals and specialty materials. Numerous techniques have been employed to improve the powder characteristics of solid dispersion products so that they are homogenous and able to accomplish the task for which they are designed unencumbered by process limitations.

Powder properties, processing procedures and requirements vary widely between industries and applications. However, all powder processing operations share a common goal —to effectively process the powder to ensure the quality and/or performance of the final product. This is especially important if the particle size of the powder is in the nano or micron range.

Advanced surface-chemical approaches and suitable technical methods and equipment are required to break up agglomerates and ensure homogeneity throughout the entire process. Traditionally, liquid-based processing methods, such as sieving and spray drying, have provided adequate conditions for mixing and homogenizing fine powders and other additives. Yet, powders processed with these techniques often do not retain their homogeneity in subsequent processing steps, such as drying and compaction. While other techniques, such as freeze granulation, have emerged as a more efficient alternative to ensuring homogeneity in the final product, these techniques are usually expensive and problems still remain with variability in the final product.

Spray drying is a common method utilized for producing a dry powder from a fluid (liquid or slurry) by rapidly drying with a hot gas. Spray dryers use some type of atomizer or spray nozzle to disperse a liquid or slurry into a controlled drop size spray. One of its main advantages is the ability to produce a consistent particle size distribution. Air is commonly used as the heated drying medium, however, other mediums (e.g., nitrogen) may be used depending on the requirements of the process. Depending on the process needs, drop sizes from 10 to 500 μm can be achieved with the appropriate choices. The most common applications are in the 100 to 200 μm diameter range. The dry powder product is often free-flowing.

Spray drying has been used to efficiently convert a liquid product into a dried powder in a single step. It advantageously provides some control over the particle formation process. A typical spray dryer uses compressed air and high temperatures to atomize liquid slurries and create dried particulate product. Generally, spray drying involves dispersing a liquid or slurry in a hot gas to produce a dry powder product. A wide range of pumpable solutions, suspensions and emulsions can be used as spray-drying feeds. A cyclone separator can be used to harvest fine particles created during the process. Spray drying allows processors to generate powders with precisely defined properties. By controlling process parameters—including the characteristics of the liquid feed, the method of atomization, the configuration of the dryer and others—chemical makers can control the shape, flow properties and porosity of the solid particles produced. Thus, by altering process parameters, one can produce complex powders that meet exact powder properties in terms of particle size and shape, bulk density, dispersibility, polymorphism, flow properties, etc., in an efficient manner. Spray drying has been applied in the production of numerous products in the chemical process industries ranging from advanced chemical compounds to bulk chemicals. Spray drying plants can be designed for almost any capacity from small quantities up to several metric tons per hour.

Spray drying is often considered a dehydration process, though it also can be used for the encapsulation of hydrophilic and hydrophobic compounds within different carriers without substantial thermal degradation, even of heat-sensitive substances. This is due to the fast drying (seconds or milliseconds) of the fluid and the relatively short exposure time(s) to heat it, which are inherent characteristics of conventional spray drying systems. The solid particles obtained present relatively narrow size distribution at the submicron-to-micron scale.

Spray drying is appealing under laboratory and industrial setups because it is a rapid, continuous, reproducible, efficient, scalable, cost-effective and single-step process. The final drying step(s) required in other common techniques used to produce particles (e.g., emulsion/solvent evaporation) is not required in spray drying. Spray drying is generally faster and cheaper than freeze-drying because it does not involve deep cooling, usually associated with great energy consumption. Due to fast solvent evaporation times and short high temperature exposure times, spray drying can be utilized to dry a broad spectrum of compounds, including heat-sensitive substances without major detrimental effects.

Spray drying has also attracted the interest of researchers to encapsulate drugs, extracts, aromatic oils, pigments and flavors within different types of carriers, such as polymeric nanoparticles (NPs), microparticles (MPs) and nanocomposites (NCs). In addition, spray drying is a processing method with pronounced inherent potential to produce pure drug particles. Spray drying can also be used to convert crystalline based products into amorphous based products. Buchi Corporation (New Castle, Del.) is one provider of industrial spraying drying equipment.

In the pharmaceutical industry, solid dispersions can be formed via chemical reactions and the use of varying approaches to produce an efficacious pharmaceutical ingredient or a dosage form of a combination of pharmaceutical ingredients. Conventional solid granulation instrumentation includes drying sieves, drying ovens, drying mills, freeze drying apparatuses, fluid bed apparatuses, etc. Enhancing the solubility and bioavailability of a solid dispersion is a challenging task in the manufacturing of pharmaceutical drugs. Spray drying is commonly used in the pharmaceutical industry to meet these tasks. Patel et. al. has reviewed the use of spray drying in several articles. See Bhavesh B. Patel, Jayvadan K. Patel, Subhashis Chakraborty, DaliShuklad, Revealing facts behind spray dried solid dispersion technology used for solubility enhancement, Saudi Pharmaceutical Journal, Volume 23, Issue 4, pages 352-365 (September 2015); and Bhavesh B. Patel, Jayvadan K. Patel and Subhashis Chakraborty, Review of Patents and Application of Spray Drying in Pharmaceutical, Food and Flavor Industry, Recent Patents on Drug Delivery & Formulation, Volume 8, pages 63-78 (February 2014). These articles note that amorphous solid dispersion based spray drying technology has been successfully used for the development of pharmaceutical products from laboratory to commercial scale. They point out that spray drying enables the drug manufacturer to impart a wide range of powder characteristics to the final product and it is often used to enhance drug delivery characteristics via solubility and bioavailability enhancement.

Once you have a solid product, it can be further processed with a conventional technique known as fluid bed technology. This technology is utilized to carry out drying, granulating and/or coating/layering of solid products via a fluid bed system. Fluid bed processing has long been used in the pharmaceutical and chemical industries as a way of enhancing the characteristics of powders, drying products and applying a coating to the surface of particles. A fluidized bed brings about the physical phenomenon that occurs when a quantity of a solid particulate substance (usually present in a holding vessel) is placed under appropriate conditions to cause a solid/fluid mixture to behave as a fluid. This is usually achieved by the introduction of pressurized fluid (liquid or gas) through the particulate medium. This results in the medium having many properties and characteristics of normal fluids, such as the ability to free-flow under gravity, or to be pumped using fluid type technologies. The resulting phenomenon is called fluidization.

Fluidization is a process similar to liquefaction, whereby a granular material is converted from a static solid-like state to a dynamic fluid-like state. Fluidization occurs when a fluid (liquid or gas) is passed up through the granular material. When fluid flow is introduced through the bottom of a bed of solid particles, it will move upwards through the bed via the empty spaces between the particles. At low fluid velocities, aerodynamic drag on each particle is also low, and thus, the bed remains in a fixed state. However, when the fluid velocity is increased, the aerodynamic drag forces will begin to counteract the gravitational forces, causing the bed to expand in volume as the particles move away from each other. Further increasing the velocity, the forces will reach a critical value at which point the upward drag forces will equal the downward gravitational forces, causing the particles to become suspended within the fluid. At the critical value, the bed is said to be fluidized and will exhibit fluidic behavior. By further increasing fluid velocity, the bulk density of the bed will continue to decrease, and its fluidization becomes more violent, until the particles no longer form a bed and are “conveyed” upwards by the fluid flow.

Notwithstanding the advantages of spray drying and fluidization, these technologies possess several significant limitations. For example, fluid bed systems are designed to process powders by drying, granulating and/or coating of the product suspended by a fluidization airstream. Accordingly, the geometry of a fluid bed is not well suited for creating solid dispersions because the processing container is not optimized to effectively create particles from sprayed droplets. Additionally the processing configuration of a fluid bed system does not allow for a cyclone separator that is often necessary to harvest the fine particles created during spray drying.

With spray drying, the yield can be detrimentally affected by the loss of product in the walls of the drying chamber and the limited capacity of the apparatus to separate fine particles. The low yields are particularly problematic during scale-up to commercial sized batches. Furthermore, recrystallization, agglomeration and non-homogeneity are common issues for all technologies that require additional measures to mediate them.

Accordingly, there remains the need for improved processes to produce solid dispersions.

The approach described in this patent seeks to address these limitations and provide a simple, reproducible and efficient process for the production of solid dispersions that are highly homogenous and better able to accomplish the task for which they are designed unencumbered by process limitations.

### SUMMARY

We describe herein a new process for producing solid dispersions, particularly, amorphous powder dispersions, utilizing a fluid bed and a carrier particle. One aspect of the invention provides a process for producing a solid dispersion of a material by spraying the material onto a fluidized cloud of carrier particles in a fluid bed. The fluidized cloud of carrier particles may be formed by adding a solid or liquid carrier to the fluid bed. The material to be sprayed is in a liquid or gas form. Thus, if the starting material is a solid, the process includes the step of liquefying or gasifying the material to facilitate the spraying of it onto the cloud of carrier particles.

In an embodiment, a process for producing a solid dispersion comprising a material and a carrier is described herein, the steps of the process comprising:
(a) fluidizing a solid or liquid carrier in a fluid bed to form a fluidized cloud of carrier particles; and
(b) spraying a liquid or gas material onto the fluidized cloud of particles to form the solid dispersion.

In a particular embodiment, a process for producing a solid dispersion comprising a pharmaceutical active ingredient and a silica carrier is described herein, the steps of the process comprising:
(a) adding a solid pharmaceutical active ingredient to a solvent to form a solution, suspension or emulsion;
(b) fluidizing the silica carrier in a fluid bed to form a fluidized cloud of silica carrier particles; and
(c) spraying the solution, suspension or emulsion onto the fluidized cloud of silica carrier particles to form the solid dispersion.

The processes described herein are simple, rapid, continuous, reproducible, efficient, scalable and cost-effective. There is no need for additional processing equipment. Yields and processing times are enhanced, while providing improved homogeneity in the final solid dispersion product.

The foregoing and other objects and features of the disclosure will become more apparent from the following detailed description, which proceeds with reference to the accompanying figures. Further embodiments, forms, features, aspects, benefits, objects and advantages of the invention shall become apparent from the detailed description and figures provided herewith.

### BRIEF DESCRIPTION OF THE DRAWINGS

The following drawings form part of the specification and are included to further demonstrate certain embodiments or various aspects of the invention. In some instances, embodiments of the invention can be best understood by referring to the accompanying drawings in combination with the detailed description presented herein. The description and accompanying drawings may highlight a certain specific example, or a certain aspect of the invention. However, one skilled in the art will understand that portions of the example or aspect may be used in combination with other examples or aspects of the invention. The features, objects and advantages will become more readily apparent when consideration is given to the detailed description below. Such detailed description makes reference to the following drawings.

FIG. 1. An illustration of a silicon dioxide particle pore volume using the processing method of Example 3.

FIG. 2. An illustration of a silicon dioxide particle pore volume using the processing method of Example 1.

FIG. 3. A photograph of the saturated solution of Example 2.

FIG. 4. X-ray powder diffraction patterns of commercial crystalline curcumin and an amorphous solid dispersion product of curcumin using the processing method of Example 5.

FIG. 5. Dissolution profiles of a commercial crystalline curcumin capsule and an amorphous solid dispersion tablet of curcumin using the processing method of Example 6.

FIG. 6. Focused beam reflectance measurement (FBRM) total count profile of a commercial crystalline curcumin capsule and an amorphous solid dispersion tablet of curcumin using the processing method of Example 6.

FIG. 7. X-ray powder diffraction pattern of an amorphous solid dispersion of curcumin using the spray drying method of Example 8.

FIG. 8. Photographs of storage stability testing for an amorphous solid dispersion product of curcumin using the processing method of Example 5 and an amorphous solid dispersion product of curcumin using the processing method of Example 8.

While the present invention is susceptible to various modifications and alternative forms, exemplary embodiments thereof are shown by way of example in the drawings and are herein described in detail. It should be understood, however, that the description of exemplary embodiments is not intended to limit the invention to the particular forms disclosed, but on the contrary, the intention is to cover all modifications, equivalents and alternatives falling within the spirit and scope of the invention as defined by the embodiments herein and the claims below. Reference should therefore be made to the embodiments and claims for interpreting the scope of the invention.

### DETAILED DESCRIPTION

We have developed a new process to create a solid dispersion without the need for spray drying equipment. The process comprises the utilization of a fluid bed system to form a fluidized cloud of carrier (e.g., silicon dioxide) particles and the spraying of a material onto the carrier cloud for the preparation of a solid dispersion comprising the material loaded onto the carrier. The process particularly described and exemplified herein is particularly useful for the production of pharmaceutical compositions. The pharmaceutical solid products produced by our process are typically substantially or fully amorphous, though partially and fully crystalline products may also be produced. Additionally, the process can be applied to any field that employs the use of a solid powder dispersion, such as foodstuffs, flavorings, dietary supplements, fragrances, cosmetics, coatings, specialty chemicals and specialty materials. The processes described herein are useful for increasing yield and processing times without the need for specialized processing equipment and/or secondary drying equipment, such as spray drying.

### Definitions

The following definitions are included to provide a clear and consistent understanding of the specification and claims. As used herein, the recited terms have the following meanings. All other terms and phrases used in this specification have their ordinary meanings as one of skill in the art would understand. Such ordinary meanings may be obtained by reference to technical dictionaries, such as Hawley's Condensed Chemical Dictionary 14^{th} Edition, by R. J. Lewis, John Wiley & Sons, New York, N.Y., 2001.

References in the specification to “one embodiment”, “an embodiment”, etc., indicate that the embodiment described may include a particular aspect, feature, structure, moiety, or characteristic, but not every embodiment necessarily includes that aspect, feature, structure, moiety, or characteristic. Moreover, such phrases may, but do not necessarily, refer to the same embodiment referred to in other portions of the specification. Further, when a particular aspect, feature, structure, moiety, or characteristic is described in connection with an embodiment, it is within the knowledge of one skilled in the art to affect or connect such aspect, feature, structure, moiety, or characteristic with other embodiments, whether or not explicitly described.

The singular forms “a,” “an,” and “the” include plural reference unless the context clearly dictates otherwise. Thus, for example, a reference to “a compound” includes a plurality of such compounds, so that a compound X includes a plurality of compounds X. It is further noted that the claims may be drafted to exclude any optional element. As such, this statement is intended to serve as antecedent basis for the use of exclusive terminology, such as “solely,” “only,” and the like, in connection with any element described herein, and/or the recitation of claim elements or use of “negative” limitations.

The term “and/or” means any one of the items, any combination of the items, or all of the items with which this term is associated. The phrase “one or more” is readily understood by one of skill in the art, particularly when read in context of its usage. For example, the phrase can mean one, two, three, four, five, six, ten, 100, or any upper limit approximately 10, 100, or 1000 times higher than a recited lower limit.

As will be understood by the skilled artisan, all numbers, including those expressing quantities of ingredients, properties such as molecular weight, reaction conditions, and so forth, are approximations and are understood as being optionally modified in all instances by the term “about.” These values can vary depending upon the desired properties sought to be obtained by those skilled in the art utilizing the teachings of the descriptions herein. It is also understood that such values inherently contain variability necessarily resulting from the standard deviations found in their respective testing measurements. When values are expressed as approximations, by use of the antecedent “about,” it will be understood that the particular value without the modifier “about” also forms a further aspect.

The terms “about” and “approximately” are used interchangeably. Both terms can refer to a variation of ± 5%, ± 10%, ± 20%, or ± 25% of the value specified. For example, “about 50” percent can in some embodiments carry a variation from 45 to 55 percent, or as otherwise defined by a particular claim. For integer ranges, the term “about” can include one or two integers greater than and/or less than a recited integer at each end of the range. Unless indicated otherwise herein, the terms “about” and “approximately” are intended to include values, e.g., weight percentages, proximate to the recited range that are equivalent in terms of the functionality of the individual ingredient, composition, or embodiment. The terms “about” and “approximately” can also modify the end-points of a recited range as discussed above in this paragraph.

As will be understood by the skilled artisan, all numbers, including those expressing quantities of ingredients, properties such as molecular weight, reaction conditions, and so forth, are approximations and are understood as being optionally modified in all instances by the term “about.” These values can vary depending upon the desired properties sought to be obtained by those skilled in the art utilizing the teachings of the descriptions herein. It is also understood that such values inherently contain variability necessarily resulting from the standard deviations found in their respective testing measurements.

As will be understood by one skilled in the art, for any and all purposes, particularly in terms of providing a written description, all ranges recited herein also encompass any and all possible sub-ranges and combinations of sub-ranges thereof, as well as the individual values making up the range, particularly integer values. A recited range (e.g., weight percentages or carbon groups) includes each specific value, integer, decimal, or identity within the range. Any listed range can be easily recognized as sufficiently describing and enabling the same range being broken down into at least equal halves, thirds, quarters, fifths, or tenths. As a non-limiting example, each range discussed herein can be readily broken down into a lower third, middle third and upper third, etc. As will also be understood by one skilled in the art, all language such as “up to”, “at least”, “greater than”, “less than”, “more than”, “or more”, and the like, include the number recited and such terms refer to ranges that can be subsequently broken down into sub-ranges as discussed above. In the same manner, all ratios recited herein also include all sub-ratios falling within the broader ratio. Accordingly, specific values recited for radicals, substituents, and ranges, are for illustration only; they do not exclude other defined values or other values within defined ranges for radicals and substituents.

One skilled in the art will also readily recognize that where members are grouped together in a common manner, such as in a Markush group, the invention encompasses not only the entire group listed as a whole, but each member of the group individually and all possible subgroups of the main group. Additionally, for all purposes, the invention encompasses not only the main group, but also the main group absent one or more of the group members. The invention therefore envisages the explicit exclusion of any one or more of members of a recited group. Accordingly, provisos may apply to any of the disclosed categories or embodiments whereby any one or more of the recited elements, species, or embodiments, may be excluded from such categories or embodiments, for example, for use in an explicit negative limitation.

The term “contacting” refers to the act of touching, making contact, or of bringing to immediate or close proximity, including at the cellular or molecular level, for example, to bring about a physiological reaction, a chemical reaction, or a physical change, e.g., in a solution, in a reaction mixture, in vitro, or in vivo.

An “effective amount” refers to an amount effective to bring about a recited effect, such as an amount necessary to form products in a reaction mixture. Determination of an effective amount is typically within the capacity of persons skilled in the art, especially in light of the detailed disclosure provided herein. The term “effective amount” is intended to include an amount of a compound or reagent described herein, or an amount of a combination of compounds or reagents described herein, e.g., that is effective to form products in a reaction mixture. Thus, an “effective amount” generally means an amount that provides the desired effect.

The term “substantially” as used herein, is a broad term and is used in its ordinary sense, including, without limitation, being largely but not necessarily wholly that which is specified. For example, the term could refer to a numerical value that may not be 100% the full numerical value. The full numerical value may be less by about 1%, about 2%, about 3%, about 4%, about 5%, about 6%, about 7%, about 8%, about 9%, about 10%, about 15%, or about 20%.

As used herein, “substantially amorphous” refers to greater that about 50% of a solid dispersion lack a specific order, shape or form. Substantially amorphous may also refer to greater than 75%, greater than 90%, or greater than about 95% of a solid dispersion, lack a specific order, shape or form. Conversely, “substantially crystalline” refers to greater that about 50% of solid crystals are of a particular crystalline form. Substantially crystalline may also refer to greater that 75%, greater than 90%, or greater than about 95% of solid crystals, are of a particular crystalline form.

The term “fluidization” (“fluidizing”) as used herein, refers to a process for the conversion of a granular material from a static solid-like state to a dynamic fluid-like state. Utilizing fluid bed technology, fluidization occurs when a fluid (liquid or gas) is passed up through the granular material. When fluidized, a bed of solid particles will behave as a fluid, like a liquid or gas. Thus, fluidization may be utilized to fluidize a solid form of a granular material. A solution, emulsion or suspension of granular material can also be fluidized in a similar manner.

The art of formulating a solid pharmaceutical composition entails the sophisticated application of multiple scientific disciplines—e.g., chemistry, biology, physics, mathematics and materials. One common challenge to the formulator is how best to mix a pharmaceutical active ingredient with an excipient, such as a carrier, to form a homogenous solid composition of the active ingredient. Often, the homogeneity of the final product is less than desired, which can produce deleterious effects.Despite considerable efforts, the loading efficiency of an active ingredient onto a carrier often leads to less than optimal results, such as significant and undesirable void volumes in the final product. Void volumes in solid dispersions are a common defect seen with traditional processes. For example, in a conventional process to mix an active ingredient with a silicon dioxide carrier, the internal pore of a single silicon dioxide carrier particle is filled with a mixture of active ingredient and solvent. When the solvent is evaporated from the particle, a void is left behind in the particle. The void volume is a measurement of the void left behind.

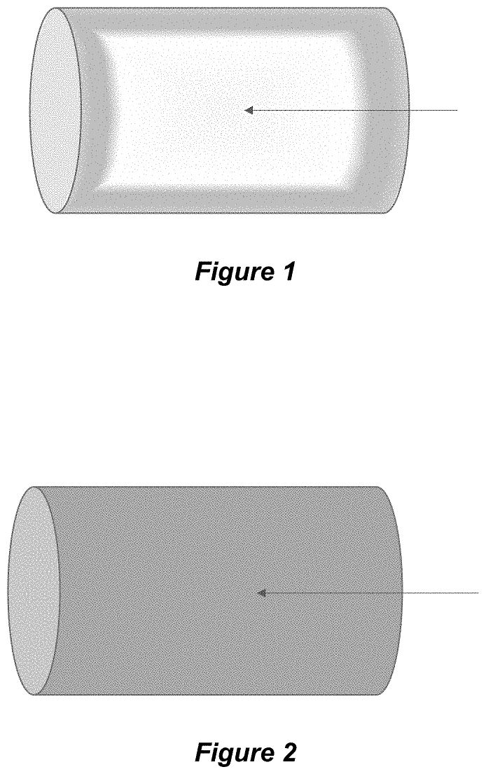

FIG. 1 provides an illustration of a void commonly seen in a conventional solid pharmaceutical composition comprising a pharmaceutical active ingredient loaded onto a carrier particle. This figure shows a void volume of greater than about 70% after solvent evaporation. This means that the active ingredient is only loaded less than about 30% onto the particle. A void volume can lead to several deleterious effects, such as tablet defects and cracking due to entrapped air in the formulation matrix. A void volume also significantly reduces the quantity of active ingredient that can be loaded onto the particle. The lack of homogeneity caused by a void volume is a serious problem for critical applications, such as delivering a drug in vivo.

We have addressed these issues by developing a new process (NucleLoad™ process) for producing a dried solid (e.g., powder) dispersion (NucleLoad™ solid dispersion) without the need of utilizing any secondary drying equipment, such as a spray dryer. The NucleLoad™ solid dispersions produced by the NucleLoad™ process exhibit enhanced physico-chemical properties by providing a more uniform loading of a material into the pores of carrier particles. This is especially useful to create a more efficient loading concentration of a pharmaceutical active ingredient. The NucleLoad™ processes described herein can efficiently load a solid active pharmaceutical ingredient into the pores of a solid carrier particle and avoid the deficiencies of conventional processes that attempt to do the same.

In an embodiment, a pharmaceutical active ingredient is dissolved into a solvent with or without additional ingredients and sprayed onto a fluidized cloud of silicon dioxide particles via the use of fluid bed technology. Our process provides for simultaneous wetting, deposition and evaporation of the processing solvent, which allows for an environment that builds up the internal pore structure over time to a substantially full particle. A substantially full pore (e.g., >95%) in a silicon dioxide particle produced by the NucleLoad™ process is illustrated in FIG. 2. This means the void volume is <5%. Compared to the relatively large void volumes commonly seen with conventional processes (FIG. 1), the small void volumes seen with the NucleLoad™ process (FIG. 2) can avoid the deleterious effects of significant void volume often seen with solid dispersion products. Utilizing the NucleLoad™ processes described herein can maximize loading efficiency, which in turn, will minimize void volumes. For example, the loading efficiency on a volume basis may be greater than 30%, greater than 50%, greater than 65%, greater than 75%, greater than 90%, greater than about 95%, or greater than 98%. Conversely, the void volume may be less than 70%, less than 50%, less than 35%, less than 25%, less than 10%, less than about 5%, or less than 2%.

A composition prepared according to our process can be readily characterized and differentiated from compositions prepared by conventional physical mixtures using standard analytical tools for determining physico-chemical properties. Providing a more homogenous composition brings numerous benefits, such as, a broader therapeutic applicability, a potentiation or synergism of the activity of each of the components of the complex, and/or other enhancement of pharmacokinetic properties.

In one aspect, the process comprises combining pharmaceutical ingredients in predefined quantities in a suitable medium or carrier and recovering the solid dispersion. In an embodiment, we teach a process for the preparation of a solid dispersion comprising the steps of: (i) forming a solution of each component (e.g., pharmaceutical active ingredient(s) and/or excipient(s)) in an appropriate solvent; (ii) mixing the solutions of step (i) together at a suitable temperature to form a single solution containing all the components; and (iii) isolating a solid dispersion of the solution of step (ii) via a conventional fluid bed. In a particular embodiment, carrier particles are fluidized in a fluid bed and the solution of step (ii) is sprayed onto the fluidized carrier particles. Alternatively, one or more (but less than all) of the individual components may be subjected to steps (ii) and (iii) and a final composition can be formulated via conventional processing afterwards. In other embodiments, an emulsion or suspension is formed instead of a solution in steps (i) and/or (ii). These processes allow one to eschew the use of a spray dryer and its attendant loss of particle into the inlet plenum. A formulator skilled in the art can readily obtain complexes of different molar ratios by adjusting the molar amounts of each component in step (i).

A pharmaceutical compound is usually combined with a suitable solid carrier, provided that the resulting combination exhibits physical properties that allow it to be more easily formulated than the parent compound. Examples of suitable solid carriers include polysaccharides and minerals or derivatives of silicon (e.g., silicate minerals), aluminum and/or magnesium. In embodiments, suitable solid carriers may comprise finely divided solids, such as kaolin, bentonite, hectorite, colloidal magnesium-aluminum silicate, silicon dioxide, magnesium trisilicate, aluminum hydroxide, magnesium hydroxide, magnesium oxide, microcrystalline cellulose, alumina, clay, talc, and the like. In other embodiments, the solid carrier can comprise calcium silicate (e.g., Zeopharm®) and/or magnesium aluminometasilicate (e.g.,)Neusilin®.

Suitable silica derivatives may include, for example, those that are described in international patent application publication number WO 03/037379 and the references cited therein, which are herein incorporated by reference. Typically, these silica derivatives comprise a granular hydrophilic fumed (and/or colloidal) silica that has a mean particle diameter of 10 to 200 microns and a BET surface area of 40 to 400 m²/g (determined according to DIN 66 131 with nitrogen). The silica derivatives also typically have a pore volume of about 0.5 to 2.8 mL/g, wherein less than about 5% of the overall pore volume has a pore diameter of less than about 5 nm, the remainder being mesopores and macropores. Additionally, the silica derivatives typically have a pH in the range of about 3.6 to about 8.5 and a tamped density of about 220 to about 700 g/L.

One specific silica material that is particularly useful in the compositions and methods described herein is AEROPERL® 300 (fumed/colloidal silica), which is available from Evonik Degussa AG (Dusseldorf, Germany). Other specific silica materials that are particularly useful are Syloid® grades available from W. R. Grace and Company (Columbia, Md.). Additionally, other materials having physical and chemical properties similar to the silica materials described herein can also be used. In embodiments of the invention, the silica particles have a mean grain diameter of 20-40 microns. In embodiments of the invention, the silica particles have a BET surface area of at least 150 m²/g, at least 200 m²/g, at least 250 m²/g, or at least 275 m²/g.

In the experimental section below, we have utilized carriers in our process having the following properties:
(a) a mean particle diameter of about 10-200 microns;
(b) a mean particle size of about 150 microns;
(c) a BET surface area of about 40-400 m²/g; and
(d) a pore volume of about 0.5-2.8 mL/g.

Using the NucleLoad™ loading processes and silica carriers described herein, we have enhanced the loading efficiency of a pharmaceutical active ingredient onto the silica carrier particles. We have determined that greater than 95% (w/w) of a pharmaceutical active ingredient or a mixture of one or more pharmaceutical active ingredients with excipients can be loaded onto the silica carrier particles. As mentioned above, a high loading capacity is particularly beneficial for pharmaceutical applications.

As revealed above, fluid bed processing has been utilized in many industries, such as pharmaceutical, foodstuff, petroleum and chemical processing. In the pharmaceutical industry, fluid bed granulation with subsequent fluid bed drying and coating, are useful methods to produce granules for pharmaceutical manufacturing. In the NucleLoad™ processes described herein, fluid bed technology is used in a novel way to enhance the loading efficiency of one or more compounds and produce a highly loaded solid dispersion product. The fluid beds can handle aqueous solutions, organic solvent solutions (i.e., acetone, isopropanol, ethanol and methanol) and/or suspensions for spraying. The specific processing parameters, selection of processing solvent(s), and ratios of ingredients can be adjusted to create the desired solid dispersion of interest. Examples of commercial fluidized beds for the pharmaceutical industry that may be utilized with the processes described herein are those produced by Robert Bosch Packaging Technology, Inc. (Minnesota, US). Bosch produces various types of nozzle spraying configurations, such as top-spray, Wurster bottom spray, tangential/radial spray and Huttlin bottom spray. The Bosch Solidlab 1 and Solidlab 2 systems are two types of fluid beds that may be utilized with our processes.

When the starting material to be used in a NucleLoad™ process is in a solid form, it can be beneficial to first transform the solid into a solution, emulsion or suspension, which is a preferred form for spraying the material into the fluid bed. Conventional methods may be employed to achieve this task, such as the application of heat or the addition of a solvent (e.g., water, acetone, ethyl acetate, dichloromethane, methanol ethanol, isopropanol or a mixture thereof).

The solid pharmaceutical dispersions produced according to the processes described herein can be used to create many different kinds of pharmaceutical formulations. The solid dispersions can be combined with pharmaceutically acceptable diluents, excipients and/or other carriers, such as lactose, povidone, croscarmellose sodium, microcrystalline cellulose, magnesium stearate, starch, sodium starch glycolate, pregelatinized starch, colloidal silicon dioxide, and the like. Pharmaceutically acceptable salts and solvates may be obtained using standard procedures well known in the art. The solid dispersions can be formulated as pharmaceutical compositions and administered to a mammalian host, such as a human patient, in a variety of forms. The forms can be specifically adapted to a chosen route of administration, e.g., oral or parenteral administration, by intravenous, intramuscular, topical or subcutaneous routes. The solid dispersions described herein may be systemically administered in combination with a pharmaceutically acceptable vehicle, such as an inert diluent or an assimilable edible carrier. For oral administration, compounds can be enclosed in hard or soft-shell gelatin capsules, compressed into tablets, or incorporated directly into the food of a patient's diet. The solid dispersions may also be combined with one or more excipients and used in the form of ingestible tablets, buccal tablets, troches, capsules, elixirs, suspensions, syrups, wafers, and the like. The amount of active ingredient in therapeutically useful compositions can be such that an effective dosage level can be obtained.

The tablets, troches, pills, capsules, and the like may also contain one or more of the following ingredients: binders, such as gum tragacanth, acacia, corn starch and/or or gelatin; excipients, such as dicalcium phosphate; disintegrating agents, such as corn starch, potato starch, alginic acid, and the like; lubricants, such as magnesium stearate; sweetening agents, such as sucrose, fructose, lactose and/or aspartame; and flavoring agents, such as peppermint, oil of wintergreen and/or cherry flavoring. When the unit dosage form is a capsule, it may contain, in addition to materials of the above type, a liquid carrier, such as a vegetable oil and/or a polyethylene glycol. Various other materials may be present as coatings or to otherwise modify the physical form of the solid unit dosage form. For instance, tablets, pills or capsules may be coated with gelatin, wax, shellac, sugar, and the like. A syrup or elixir may contain a pharmaceutical active compound with one or more inactive ingredients, such as sucrose and/or fructose as a sweetening agent, methyl and/or propyl parabens as a preservative, and a dye and/or flavoring, such as cherry or orange flavor. Any material used in preparing a unit dosage form should be pharmaceutically acceptable and substantially non-toxic in the amounts employed. In addition, the active compound may be incorporated into sustained-release preparations and devices.

Formulations may be prepared by conventional procedures well known in the pharmaceutical art. It will be appreciated that the pharmaceutical compositions described herein may be varied according to well-known pharmaceutical techniques to accommodate differing amounts and types of active ingredient. Additionally, the specific ingredients and proportions described herein are for illustrative purposes. Ingredients may be exchanged for suitable equivalents and proportions may be varied, according to the desired properties of the dosage form of interest.

A skilled artisan in the field of pharmaceutical formulations can utilize the NucleLoad™ processes described herein to form a high quality solid dispersion, then routinely incorporate the solid dispersion in an appropriate formulation depending on the requirements for product.

The following Examples are intended to illustrate the above invention and should not be construed as to narrow its scope. One skilled in the art will readily recognize that the Examples suggest many other ways in which the invention could be practiced. It should be understood that numerous variations and modifications may be made while remaining within the scope of the invention.

### EXAMPLES

### Example 1

### Loading of Acetaminophen on Silicon Dioxide Using Bosch Solidlab 1 Fluid Bed (NucleLoad™ Process)

An acetaminophen solution was prepared by dissolving 80.25 g of solid acetaminophen (APAP) in 375 g methanol.

Density of methanol is 0.791 g/mL.

Solution volume is 474 mL.

Solution concentration on a volume basis is 80,250 mg/474 mL=169 mg APAP/mL.

Solution concentration on a mass basis is 80.25 g/(80.25 g+375 g)=0.1763 g APAP/g solution.

Air was used as the pressurized fluid in the Bosch Solidlab 1 fluid bed.

Added 75.73 g of silicon dioxide (Grace Syloid® XDP-3150, 150 μm particle size) to the Bosch Solidlab 1 fluid bed to form a fluidized cloud of silicon dioxide carrier particles for the processing of a NucleLoad™ solid dispersion. The bottom spray configuration of the fluid bed was used to spray the acetaminophen solution onto the fluidized cloud of silicon dioxide carrier particles.

| | Product | Inlet | Outlet | | | | atomization | pattern air |
|---|---|---|---|---|---|---|---|---|
| Time | Temp | Temp | Temp | airflow | spray | | pressure | pressure |
| (min) | (° C.) | (° C.) | (° C.) | (m\hat{ }3/hr) | setting | % LEL | (bar) | (mbar) |
| 0 | 31.7 | 45.7 | 27.3 | 10 | 20 | 0.0 | 0.7 | 427 |
| 5 | 31.1 | 52.6 | 28.1 | 10 | 20 | 4.9 | 0.7 | 427 |
| 10 | 32.3 | 50.3 | 28.5 | 10 | 20 | 3.3 | 0.7 | 427 |
| 15 | 32.4 | 50.0 | 28.6 | 10 | 10 | 6.8 | 0.7 | 427 |
| 20 | 32.8 | 50.0 | 29.0 | 10 | 10 | 6.0 | 0.7 | 427 |
| 26 | 37.4 | 49.7 | 31.5 | 20 | 15 | 7.2 | 0.7 | 427 |
| 31 | 35.4 | 43.5 | 31.6 | 15 | 12 | 5.6 | 0.7 | 427 |
| 36 | 33.7 | 41.6 | 30.9 | 15 | 15 | 7.1 | 0.7 | 427 |
| 40 | 33.0 | 41.9 | 30.4 | 15 | 15 | 9.8 | 0.7 | 427 |
| 45 | 32.8 | 42 | 30.2 | 15 | 15 | 9.8 | 0.7 | 427 |
| 50 | 32.7 | 42.6 | 30.2 | 15 | 18 | 12.5 | 0.7 | 427 |
| 55 | 33.3 | 45.4 | 29.9 | 15 | 18 | 13.5 | 0.7 | 427 |
| 60 | 33.3 | 45 | 29.8 | 15 | 20 | 16.3 | 0.7 | 427 |
| 65 | 33.8 | 44.5 | 30.0 | 18 | 20 | 14.1 | 0.88 | 624 |
| 70 | 35.7 | 45 | 30.1 | 18 | 22 | 15.3 | 0.88 | 624 |
| 75 | 36.3 | 45 | 30.4 | 18 | 22 | 15.3 | 0.88 | 624 |
| 80 | 35.6 | 40.5 | 29.9 | 18 | 22 | 15.3 | 0.88 | 624 |
| 85 | 34.2 | 39.7 | 29.4 | 18 | 22 | 15.3 | 0.88 | 624 |
| 90 | 33.6 | 39.9 | 29.2 | 18 | 22 | 15.5 | 0.88 | 624 |
| 95 | 33.2 | 40.0 | 29.2 | 18 | 22 | 15.3 | 0.88 | 624 |
| 97 | 33.3 | 40.0 | 29.2 | 18 | 22 | 15.2 | 0.88 | 624 |
| 100 | 34.3 | 47.2 | 31.4 | 18 | 0 | 0 | 0.88 | 624 |
| 103 | 37 | 51.3 | 33.4 | 18 | 0 | 0 | 0.88 | 624 |

Collected 103.47 g net weight of a dry solid dispersion product (NucleLoad™ solid dispersion).

The material was free flowing and suitable for further processing.

Calculation of solution sprayed:
146.00 g remaining.
455.25 g starting quantity.
309.25 g sprayed.
309.25 g×0.1763 g APAP/g=54.52 g APAP sprayed.

Yield=100%×[103.47 g/(75.73 g+54.52 g)]=79.4%.

| | convert to mL | conc | qty | | Theoretical |
|---|---|---|---|---|---|
| g | (0.791 g/mL | 0.169 | APAP | qty | loading (%) |
| sprayed | density) | (g/mL) | (g) | Syloid ® | on a mass basis |
| 309.25 | 244.62 | 0.169 | 41.34 | 75.73 | 35.31 |

Labeled sample collected as lot 15116-4A (NucleLoad™ solid dispersion). The resulting theoretical loading is based on an assay of the solid APAP loaded on a mass basis. To calculate the theoretical loading on a volume basis, solid APAP has a density of approximately 1.3 g/mL. 41.34 g of APAP would occupy a volume of 53.74 mL. Syloid® silica pore volume is 1.7 mL/g, so 75.73 g of Syloid® would have a pore volume of 128.74 mL. Theoretical loading volume=100×53.74/128.74=42% on a volume basis.

FIG. 2 shows an illustration of a silicon dioxide pore using the NucleLoad™ processing method of Example 1. The pore is substantially filled. The void volume (based on mass) was <5%.

### Example 2

### Comparative Example A

### Evaluate the Processability of Adding the Same Ratio of Ingredients as was Performed in Example 1, but Without the Use of the Fluid Bed

Added 22.02 g of an acetaminophen/methanol solution to 7.5 g silicon dioxide (Grace Syloid® XDP-3150-150 μm particle size). The silicon dioxide/acetaminophen/methanol solution became saturated (and particles came out of solution). This material was not processable (and could not be fluidized in a fluid bed).

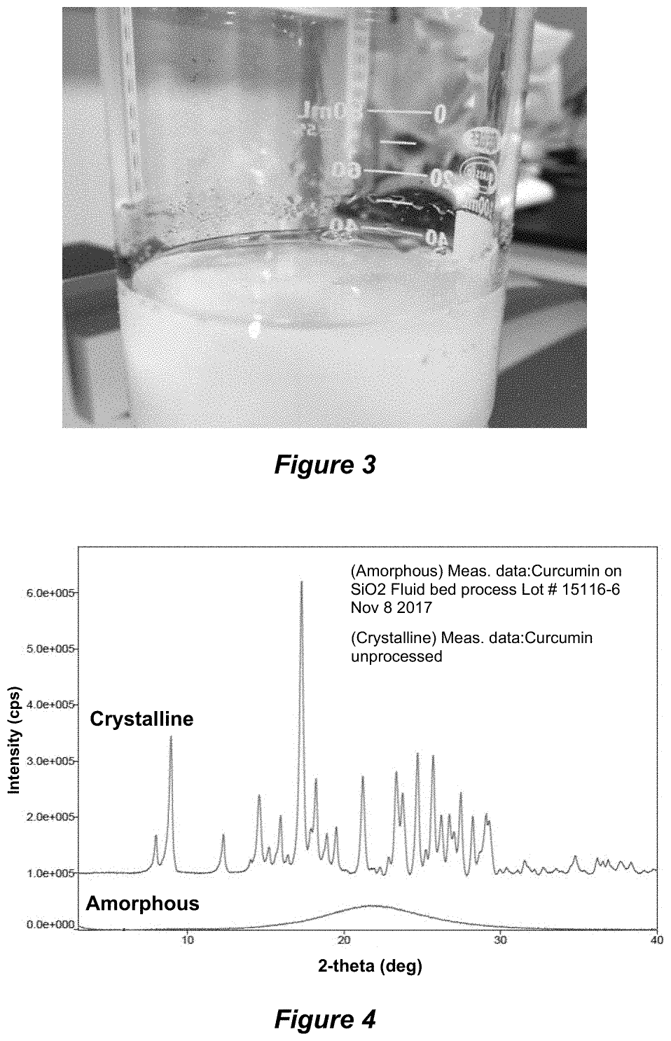

FIG. 3 shows a photograph of the saturated solution made with the processing method of Example 2.

### Example 3

### Comparative Example B

### Calculate Loading Efficiency of Example 2 Based on Pore Volume

The following example was performed at 1/10^{th} of the scale of Example 1 for comparative purposes.

7.5 g of Syloid® XDP-3150 contains 1.7 mL pore volume per gram=12.75 mL.

12.75 mL to fill with 0.791 g/mL density solution=10.09 g theoretical solution volume.

Added 10.78 g of an acetaminophen/methanol solution to the Syloid® XDP-3150 and a wet paste was formed.

Dried the paste in an oven on a pan at 60° C. overnight (approx. 15-16 hours).

Labeled sample as 15116-4C (conventional process solid dispersion).

FIG. 1 shows a picture of a silicon dioxide pore using the conventional processing method of Example 3. The evaporated solvent left a partially filled pore. The void volume (based on mass) was >50%.

A comparison of FIG. 1 (void volume in Example 3 conventional solid dispersion) to FIG. 2 (void volume in Example 1 NucleLoad™ solid dispersion) shows the superior loading efficiency of the NucleLoad™ process.

### Theoretical Loading Conc.

| Soln conc (mg/mL) | Soln vol (mL) | Qty APAP (mg) | Solids Loading % |
|---|---|---|---|
| 169 | 12.75 | 2154.75 | 22.32 |

To calculate the theoretical loading on a volume basis, solid APAP has a density of approximately 1.3 g/mL. 2.154 g of APAP would occupy a volume of 2.80 mL. Syloid® pore volume is 1.7 mL/g, so 7.5 g of Syloid® would have a pore volume of 12.75 mL. Theoretical loading volume=100×2.154/12.75=17% on a volume basis.

### Example 4

### Analysis of Sample Content of Example 1 (NucleLoad™ Solid Dispersion) vs. Example 3 (Conventional Solid Dispersion) by Denovix UV-Vis Spectrometer

| Weight of | volume of | APAP |
|---|---|---|
| APAP (mg) | methanol (mL) | Concentration |
| 3.0 | 1.5 | 2 |
| 0.5 mL of above = 1 mg | 0.5 | 1 |
| 0.5 mL of above = 0.5 mg | 0.5 | 0.5 |
| 0.5 mL of above = 0.25 mg | 0.5 | 0.25 |

| Reference Curve from Denovix UV-Vis Detector | | | | | | | |
|---|---|---|---|---|---|---|---|
| mg/mL | 248 nm | 249 nm | 250 nm | | | | |
| 0.25 | 13.27 | 13.26 | 13.16 | | | | |
| 0.5 | 31.65 | 31.66 | 31.51 | | | | |
| 1 | 59.98 | 60.03 | 59.62 | | | | |
| 2 | 123.79 | 123.83 | 123.15 | | | | |
| | | Solve for | | | | | |
| | | quantity | | | | % measured | |
| | | using 249 | mL | | mg | mass loading | |
| Sample | 249 nm | nm | solvent | mg | sample | concentration | |
| Example 1 | 80.87 | 1.32 | 4 | 5.26 | 18 | 29.25 | 1.44 increase |
| 15116-4A | | | | | | | over |
| | | | | | | | 15116-4C |
| Example 3 | 42.58 | 0.70 | 4 | 2.82 | 13.9 | 20.25 | |
| 15116-4C | | | | | | | |

The % measured loading concentration on a mass basis for Example 1 was 29.25%, which is considerably higher than the 20.25% measured loading concentration for Example 3. This is a significant finding because the NucleLoad™ process used in Example 1 achieved a 44% increase in the loading efficiency of acetaminophen over the conventional method used in Example 3, where the pore volume of the silicon dioxide limits the quantity of solvent and solids that can be added. As mentioned above, the volume occupied by the solvent after it evaporates creates a void space, which can increase the amount of entrapped air in a formulation. Entrapped air in a solid dosage formulation has been attributed to tableting defects, such as splitting and capping of the tablet matrix. The NucleLoad™ process of Example 1 minimized entrapped air by simultaneously removing the solvent while adding solid to the silicon dioxide pore volume.

### Example 5

### Loading of Curcumin on Silicon Dioxide Using Bosch Solidlab I Fluid Bed

Solid crystalline curcumin (PureBulk, Inc., Roseburg, Oreg.) was purchased for use as a raw material. The process described in this example was used to create a NucleLoad™ intermediate (NLI) comprising amorphous curcumin, which was further processed into a tablet by downstream processes. The NLI was isolated prior to the additional processing steps.

Composition of a curcumin solution is provided in the table below.

| | | % | 600 g | Actual |
|---|---|---|---|---|
| Ingredient | Comments | w/w | batch | Dispensed (g) |
| Curcumin | Curcumin Extract Lot | 1 | 6 | 6.03 |
| | 20170816-03-0250g | | | |
| Solutol ® HS15 | | 5 | 30 | 30.62 |
| Plasdone ™ K-29/32 | | 4 | 24 | 24.01 |
| Ethanol | | 90 | 540 | 602.4 |
| | | 100 | | |

Added 6.03 g curcumin, 549.5 g ethanol and 30.62 Solutol® HS15 (Kolliphor® HS15) (BASF Pharmaceuticals) (BASF USA, Florham Park, N.J.) into a 1-L media bottle, and stirred the composition on a magnetic stir plate.
Start stirring: 2:45 pm, added 52.9 g ethanol until 2:49 pm.
Start sonicating: 3:04 pm.
Stop sonicating: 3:24 pm.

Added Plasdone K-29/32 (Ashland Inc., Covington, Ky.).
Start mixing: 3:25 pm.
Stop mixing: 3:49 pm.

Air was used as a pressurized fluid in the Bosch Solidlab 1 fluid bed.

Added 35.07 g of silicon dioxide (Grace Syloid® XDP-3150-150 μm particle size) to the Bosch Solidlab 1 fluid bed to form a fluidized cloud of silicon dioxide carrier particles for the NucleLoad™ processing of a solid dispersion. Used the bottom spray configuration to spray the curcumin solution onto the fluidized cloud of silicon dioxide carrier particles.

| | | | | | | | | | pattern |
|---|---|---|---|---|---|---|---|---|---|
| | Product | Inlet | Outlet | | spray | | Spray | atomization | air |
| Time | Temp | Temp | Temp | airflow | setting | % | amount | pressure | pressure |
| (min) | (° C.) | (° C.) | (° C.) | (m\hat{ }3/hr) | (%) | LEL | (g) | (bar) | (mbar) |
| 0 | 44.1 | 49.5 | 39.3 | 34 | 20 | 0 | 0 | 0.84 | 649 |
| 5 | 45.0 | 54.2 | 38.8 | 33 | 20 | 4.1 | 13.20 | 0.84 | 647 |
| 8 | 48.2 | 60.2 | 40.5 | 33 | 30 | 9.4 | 23.46 | 0.84 | 648 |
| 10 | 48.6 | 60.9 | 40.6 | 32 | 40 | 11.2 | 29.70 | 0.84 | 649 |
| 14 | 49.0 | 60.9 | 40.3 | 31 | 40 | 17.9 | 56.70 | 1 | 714 |
| 18 | 49.5 | 60.9 | 40.3 | 30 | 50 | 24.0 | 81.75 | 1 | 712 |
| 20 | 50.5 | 64.2 | 39.7 | 29 | 50 | 26.2 | 98.97 | 1 | 712 |
| 25 | 52.2 | 64.6 | 406 | 34 | 50 | 20.8 | 143.55 | 1 | 709 |
| 29 | 51.7 | 64.5 | 41.5 | 34 | 55 | 24.9 | 174.72 | 1 | 707 |
| 30 | 51.5 | 64.7 | 41.5 | 34 | 55 | 24.1 | 182.23 | 1 | 709 |
| 34 | 51.9 | 65.2 | 41.9 | 34 | 60 | 26.3 | 225.19 | 1 | 707 |
| 35 | 51.7 | 65.2 | 41.6 | 34 | 60 | 25.3 | 240.93 | 1 | 707 |
| 40 | 51.6 | 65.0 | 41.6 | 34 | 60 | 28.2 | 281.00 | 1 | 707 |
| 45 | 54.6 | 60.7 | 47.1 | 34 | 60 | 0 | 297.70 | 1 | 704 |
| 50 | 50.9 | 59.7 | 42.6 | 34 | 60 | 16.2 | 328.99 | 1 | 706 |
| 55 | 50.0 | 59.8 | 42.2 | 34 | 60 | 28.4 | 371.90 | 1 | 704 |
| 60 | 49.9 | 60.1 | 40.9 | 34 | 60 | 27.6 | 411.10 | 1 | 704 |
| 65 | 52.0 | 63.2 | 41.7 | 34 | 60 | 27.1 | 463.31 | 1 | 703 |
| 70 | 53.3 | 63.1 | 42.0 | 34 | 60 | 26.6 | 513.64 | 1 | 705 |
| 75 | 53.6 | 63.0 | 42.2 | 34 | 60 | 26.6 | 561.29 | 1 | 703 |
| 80 | 53.4 | 63.1 | 42.6 | 34 | 60 | 26.2 | 618.09 | 1 | 703 |
| 86 (stop) | 53.1 | 63.0 | 43.5 | 34 | 60 | 0 | 656.67 | 1 | 703 |
| Dried for 5 minutes then | | | | | | | | | |
| turned off to cool. | | | | | | | | | |
| Cool in | | | | | | | | | |
| chamber | | | | | | | | | |
| overnight | | | | | | | | | |

Collected 71.96 g of NucleLoad™ curcumin amorphous intermediate out of 95 g theoretical.

Yield is 100×71.96/95=75.75%.

Standard solutions were generated by dissolving solid crystalline curcumin (PureBulk) in methanol and measuring the solutions on a Denovix UV-Vis detector to generate a calibration curve to determine curcumin concentrations in the samples. Started with most concentrated by adding 1.1 mg into 1.0 mL of methanol. Pipetted 500 μL of 1.1 mg/mL solution into a new HPLC vial and diluted with 500 μL of methanol to create 0.55 mg/mL solution. Repeated successive dilution scheme as detailed in the table below.

| Curcumin conc | Absorbance at |
|---|---|
| (mg/mL) | 425 nm |
| 0.06875 | 9.7935 |
| 0.1375 | 21.4291 |
| 0.275 | 38.0285 |
| 0.55 | 64.01 |
| 1.1 | 116.2395 |

A calibration curve for dissolved crystalline curcumin UV absorbance (Abs) as a function of concentration was calculated from the equation of conc=0.0099×Abs−0.0671 with an R-squared coefficient of 0.99532. This equation was used for the samples in table below.

### Determined Concentrations in the Samples Below.

| | Conc = Abs × 0.0099 − 0.0671 | |
|---|---|---|
| | | Concentration |
| | Abs at 425 nm | (mg/mL) |
| Crystalline curcumin | 0.0658 | Below limit of |
| in water t = 1 hr | | quantitation |
| NucleLoad ™ | 134.6935 | 1.27 |
| Intermediate of curcumin | | |
| (Example 5) in water t = 1 hr | | |
| 50:50 NucleLoad ™ Inter- | 64.889 | 0.575 |
| mediate of curcumin (Example | | |
| 5) in water | | |
| 28.8 mg NucleLoad ™ Inter- | 74.37 | 0.669 (or 1.67 mg in |
| mediate of curcumin (Example | | 2.5 mL) |
| 5) in 2.5 mL methanol | | |
| concentration | | |
| NucleLoad ™ Intermediate of | 24.6 | 0.18 |
| curcumin (Example 5) in water | | |
| 20 hour | | |
| Crystalline curcumin in water | 0.1945 | Below limit of |
| 20 hour | | quantitation |

Theoretical concentration of NucleLoad™ Intermediate is 6.316%.

Actual measured % concentration is 1.67 mg/28.8 mg=5.800%.

Adjusted Assay is 5.800%/6.316%=91.8%.

FIG. 4 shows X-ray powder diffraction patterns of a commercial (PureBulk) solid crystalline curcumin and an amorphous solid dispersion of curcumin using the NucleLoad™ processing method of Example 5. This figure confirms the amorphous nature of the curcumin NLI as compared to the commercial crystalline curcumin used in the preparation of the NLI.

### Example 6

### Formulation of an Amorphous NucleLoad™ Final Product from the NLI of Example 5 and Compression Into 24 mg Tablets

The formulation composition is provided in the table below.

A physical mixture was prepared by blending all the ingredients, except for magnesium stearate, for 5 minutes at 25 rpm.

Added magnesium stearate and blended for an additional 3 minutes at 25 rpm to form an amorphous NucleLoad™ final blended product. The NucleLoad™ final blended product exhibited good flow properties, without the need for additional granulation processing, such as roller compaction. This was advantageous with respect to processing cycle times and efficiency.

| | | | Adjust for 91.8% | | | |
|---|---|---|---|---|---|---|
| | Theoretical | | assay for 24.3 mg dose | | | |
| | Concentration | | | | qty for | Actual |
| | % | mg/ | % | mg/ | 25 g | Qty |
| Ingredient | w/w | tablet | w/w | tablet | batch | Weighed |
| Example 5 NLI | 76.0 | 380 | 83.924 | 419.6 | 20.98 | 20.98 |
| Mixture | | | | | | |
| Microcrystalline | 21.0 | 105 | 13.076 | 65.4 | 3.27 | 3.27 |
| Cellulose (Avicel | | | | | | |
| PH102) | | | | | | |
| Croscarmellose | 2.0 | 10 | 2.000 | 10.0 | 0.50 | 0.51 |
| Sodium (Ac-Di-Sol) | | | | | | |
| Mg Stearate | 1.0 | 5 | 1.000 | 5.0 | 0.25 | 0.25 |
| (Ligamed) | | | | | | |
| | 100.0 | 500 | 100 | 500 | 25 | 25.01 |

Compressed the NucleLoad™ final blended product into 24 mg tablets using a Korsch PH100 rotary tablet press. Labeled sample as lot 15116-6.

### Equipment for Analyzing Tablets.

Analytical balance for weight determination; Mitutyo micrometer for tablet thickness; and Vankel VK200 hardness tester.

| Tablet compression | weight (mg) | thickness (mm) | hardness (kp) |
|---|---|---|---|
| (7.5 × 17 mm tooling) | (n = 10) | (n = 10) | (n = 5) |
| Average | 506.1 | 5.77 | 5.3 |
| Std Dev | 5.8 | 0.0 | 0.5 |
| RSD % | 1.1 | 0.4 | 9.8 |

### Example 7

### Comparison of NucleLoad™ Amorphous Curcumin 24 mg Tablet to a Commercial Crystalline Curcumin 200 mg Capsule—Dissolution and Disintegration

The dissolution and disintegration of the NucleLoad™ curcumin tablet (lot 15116-6) of Example 6 (24 mg) was simultaneously monitored with a Focused Beam Reflectance Measurement (FBRM) device (METTLER TOLEDO Lasentec, Model S400) and a UV detection device (Denovix) at 415 and 425 nm.

Filled a 100 mL beaker with 60.1 g purified water.

Added tablet to the water with a mesh tablet holder to the beaker on the Model S400 FBRM fixed beaker stand. Simultaneously measured FBRM with fine electronics at 2 m/s scan speed while pulling samples for dissolution analysis.

Analyzed solution concentration by UV absorption with Denovix UV-Vis detector. A slight shift in the peak of curcumin to 415 nm in water was observed. Used 415 nm for calculations. 425 nm values were recorded for comparison.

| Time | | | Concentration | Quantity of Curcumin |
|---|---|---|---|---|
| (min) | 425 nm | 415 nm | Calc. (mg/mL) | in 60.1 mL water |
| 5 | 8.4569 | 8.6145 | 0.018 | 1.093 |
| 10 | 16.4082 | 16.4916 | 0.096 | 5.780 |
| 15 | 20.9786 | 21.085 | 0.142 | 8.513 |
| 20 | 24.3282 | 24.3327 | 0.174 | 10.445 |
| 25 | 24.1572 | 24.0697 | 0.171 | 10.288 |
| 30 | 23.4813 | 23.5672 | 0.166 | 9.990 |

The dissolution and disintegration of the NucleLoad™ amorphous curcumin product produced by Example 6 will now be compared to the dissolution and disintegration of a commercially available capsule formulation containing 200 mg of crystalline curcumin with turmeric essential oil. The tradename of a commercial capsule is CuraMed® (manufacturer lot 170501) and can be purchased from Terry Naturally (Green Bay, Wis.). Simultaneous monitoring was accomplished via FBRM and UV detection at 415 and 425 nm.

Filled a 100 mL beaker with 60.4 g purified water.

Added capsule to the water with a mesh tablet holder to the beaker on the Model S400 FBRM fixed beaker stand.

Simultaneously measured FBRM with fine electronics at 2 m/s scan speed while pulling samples for dissolution analysis.

Analyzed solution concentration by UV absorption with Denovix UV-Vis detector. A slight shift in the peak of curcumin to 415 nm in water was observed. Used 415 nm for calculations. 425 nm values were recorded for comparison.

The CuraMed® commercial capsule formulation (200 mg of curcumin) was advertised with claims to be “up to 500 times stronger than tumeric” and was characterized as providing “superior absorption curcumin”.

| Time | | | Concentration Calc | Quantity of Curcumin |
|---|---|---|---|---|
| (min) | 425 nm | 415 nm | (mg/mL) | in 60.1 mL water |
| 5 | −0.0542 | -0.0312 | below limit of | below limit of |
| | | | quantitation | quantitation |
| 10 | 0.3260 | 0.4932 | below limit of | below limit of |
| | | | quantitation | quantitation |
| 15 | −0.1279 | −0.1344 | below limit of | below limit of |
| | | | quantitation | quantitation |
| 20 | −0.2605 | −0.1112 | below limit of | below limit of |
| | | | quantitation | quantitation |
| 25 | 1.3023 | 1.2211 | below limit of | below limit of |
| | | | quantitation | quantitation |
| 30 | 0.2232 | 0.1916 | below limit of | below limit of |
| | | | quantitation | quantitation |

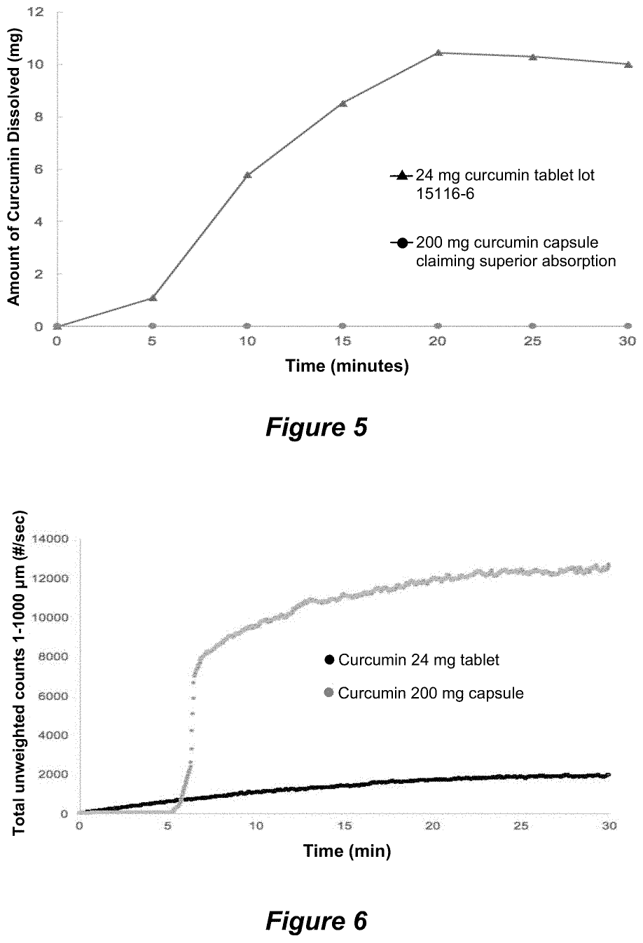

FIG. 5 shows the dissolution profiles of the commercial CuraMed® crystalline curcumin (200 mg) capsule of Example 7 and the NucleLoad™ solid dispersion amorphous curcumin (24 mg) tablet of Example 6. A comparison of the dissolution profiles of the formulations shows there was a significant increase in dissolution of curcumin for the NucleLoad™ solid dispersion tablet.

FIG. 6 shows a focused beam reflectance measurement (FBRM) total count profile of the commercial CuraMed® crystalline curcumin (200 mg) capsule of Example 7 and the NucleLoad™ solid dispersion amorphous curcumin (24 mg) tablet of Example 6. The FBRM analysis shows distinct property differences between the two formulations. The commercial CuraMed® curcumin capsule formulation exhibited a higher particle count, indicating lack of dissolution of particles. In contrast, the NucleLoad™ solid dispersion curcumin tablet exhibited a lower particle count, indicating more soluble particles, which can enhance performance characteristics.

A table showing binned chord length FBRM statistics for NucleLoad™ solid dispersion amorphous curcumin 24 mg tablets at 5 minute intervals is provided below.

| | 00:05:03 | 00:10:03 | 00:15:01 | 00:20:03 | 00:25:03 | 00:30:03 |
|---|---|---|---|---|---|---|
| Trend | (Fine) | (Fine) | (Fine) | (Fine) | (Fine) | (Fine) |
| Mean No Wt | 37 | 36 | 36 | 34 | 34 | 33 |
| Mean Lth Wt | 81 | 84 | 78 | 81 | 79 | 76 |
| counts No | 576 | 1084 | 1388 | 1698 | 1834 | 1895 |
| Wt 1-1000 | | | | | | |
| counts No | 176 | 344 | 428 | 538 | 593 | 631 |
| Wt 1-10 | | | | | | |
| counts No | 247 | 468 | 609 | 768 | 821 | 837 |
| Wt 10-50 | | | | | | |
| counts No | 120 | 211 | 271 | 302 | 329 | 336 |
| Wt 50-110 | | | | | | |
| counts No | 32 | 59 | 79 | 88 | 89 | 90 |
| Wt 110-300 | | | | | | |
| counts No | 0 | 2 | 1 | 2 | 2 | 1 |
| Wt 300-1000 | | | | | | |

A table of binned chord length FBRM statistics for commercial CuraMed® crystalline curcumin 200 mg capsules at 5 minute intervals is provided below.

| | 00:05:09 | 00:10:00 | 00:15:00 | 00:20:04 | 00:25:04 | 00:30:02 |
|---|---|---|---|---|---|---|
| Trend | (Fine) | (Fine) | (Fine) | (Fine) | (Fine) | (Fine) |
| Mean No Wt | 19 | 33 | 31 | 30 | 29 | 29 |
| Mean Lth Wt | 69 | 66 | 67 | 64 | 62 | 59 |
| counts No | 36 | 9539 | 11130 | 12004 | 12408 | 12596 |
| Wt 1-1000 | | | | | | |
| counts No | 14 | 1724 | 2158 | 2456 | 2732 | 2610 |
| Wt 1-10 | | | | | | |
| counts No | 21 | 6116 | 7143 | 7710 | 7884 | 8254 |
| Wt 10-50 | | | | | | |
| counts No | 0 | 1410 | 1520 | 1549 | 1535 | 1482 |
| Wt 50-110 | | | | | | |
| counts No | 0 | 277 | 290 | 272 | 239 | 236 |
| Wt 110-300 | | | | | | |
| counts No | 0 | 12 | 19 | 17 | 19 | 13 |
| Wt 300-1000 | | | | | | |

A comparison of the tables for the two formulations shows that the CuraMed® crystalline capsule formulation exhibited a higher count for each particle range. This further supports our analysis that the NucleLoad™ solid dispersion amorphous curcumin tablet formulation provides significantly improved solubility of the contents over time.

### Example 8

### Spray Drying of a Curcumin Formulation Using a Buchi Spray Dryer

Commercial crystalline curcumin (PureBulk) was purchased for use as a raw material. The process described in this example is used to create a spray dried amorphous intermediate from a commercial crystalline curcumin starting material. The intermediate was isolated prior to additional processing steps to produce a tablet or capsule formulation.

The composition of a curcumin spray solution is provided in the table below.

| | | | | | Actual | Bottle + |
|---|---|---|---|---|---|---|
| | | | % | 60 g | Dispensed | stir bar |
| | Lot | Expiration | w/w | batch | (g) | Tare (g) |
| Curcumin | Curcumin Extract | | 0.91 | 0.546 | 0.5460 | 170.12 |
| | Lot 20170816- | | | | | |
| | 03-0250g | | | | | |
| Solutol ® | Kolliphor ® HS-15 | | 4.62 | 2.772 | 2.77 | |
| HS15 | (Sigma) | | | | | |
| Plasdone ™ | 1590176 | — | 3.62 | 2.172 | 2.1721 | |
| K-29/32 | | | | | | |
| Reagent | 17E176504. | 04/2022 | 90.85 | 54.51 | 54.52 | |
| Alcohol | | | | | | |
| (Ethanol) | | | | | | |
| Total Solids | | | 9.15 | 5.49 | 60.01 | |

Added 0.5460 g curcumin into 54.52 g of ethanol and mixed for 10 minutes.

Added 2.1721 g of Plasdone™ K29/32 to solution.

Mixed on a stir plate for 6 minutes.

Solution became a clear bright orange solution.

Buchi spray drying with a two-fluid nozzle was performed using the processing parameters in the table below.

| | | Actual | | | | | | Final bottle |
|---|---|---|---|---|---|---|---|---|
| | Set Inlet | Inlet | Outlet | Pump | | Spray Gas | Bottle + | after |
| Time | Temp. | Temp. | Temp. | Rate | Ballance | Flow | material | spraying |
| (hh:mm:ss) | (° C.) | (° C.) | (° C.) | (%) | (g) | (mm) | (g) | (g) |
| 00:00:00 | 130 | 130 | 68 | 20 | 0 | 40 | 229.56 | 169.80 |
| 00:01:00 | 130 | 131 | 68 | 20 | 8.58 | | | |
| 00:02:00 | 130 | 129 | 68 | 20 | 11.43 | | 0.8748 | 15.93 |
| 00:03:00 | 130 | 129 | 68 | 20 | 15.25 | | | |
| 00:04:00 | 135 | 129 | 69 | 20 | 18.12 | | | |
| 00:05:00 | 135 | 136 | 72 | 20 | 26.34 | | | |
| 00:06:00 | 130 | 131 | 68 | 20 | 8.09 | 40 | | |
| 00:08:00 | 130 | 130 | 69 | 20 | — | | | |
| 00:10:10 | 130 | 130 | 71 | 15 | — | | | |

A significant amount of material adhered to the side walls of the cyclone chamber was observed. The material on the walls was a dark blood orange. The material in the collection vessel was bright orange. Scraped off material on the walls of the cyclone and mixed it in with the material deposited in the collection vessel. Dried the collected powder in a 60° C. oven overnight (approximately 16 hours) to remove any residual solvent.

The yield is calculated below.

| | |
|---|---|
| Total Container Weight Before Spraying (g) | 229.56 |
| Total Container Weight After Spraying (g) | 169.80 |
| Amount Sprayed (g) | 59.76 |
| Solids Loading | 0.07 |
| Wet Recovered Product (g) | 0.8748 |
| Wet Yield (%) | 15.9% |
| Dry Recovered Product (g) | 0.8562 |
| Dry Yield (%) | 15.6% |
| % Loss of Drying | 2.1% |

A 15.6% dry yield is consistent for the spray drying process and reflects the observation whereby a significant amount of product was stuck to the processing chamber.

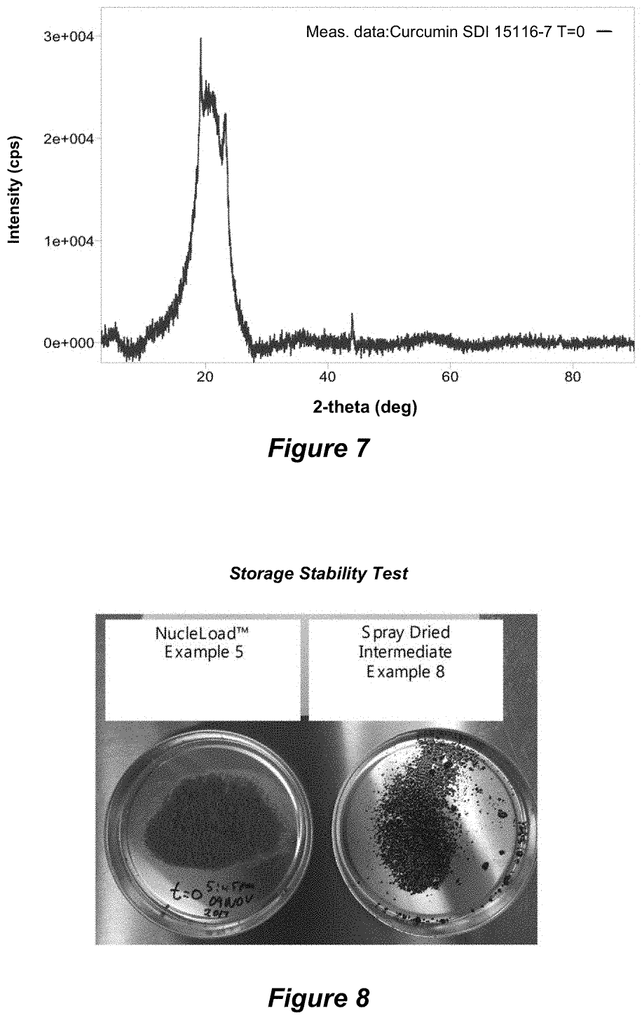

FIG. 7 provides an X-ray powder diffraction pattern of the amorphous solid dispersion of curcumin spray dried intermediate using the processing method of Example 8.

As shown in the figure, there are sharp peaks around 19 and 23 2-theta degrees indicating the presence of crystalline material in the sample. This is an important finding because spray drying (a common method of producing amorphous dispersions) did not fully convert the crystalline curcumin into an amorphous form. In contrast, the novel NucleLoad™ process detailed in Example 5 converted the curcumin into a substantially full amorphous powder (FIG. 4) with the added benefit of providing a significantly higher yield than the spray drying process of Example 8.

### Example 9

### Storage Stability Testing of Example 5 and Example 8

The amorphous curcumin solid dispersions of Example 5 and Example 8 were stored and tested for stability.

FIG. 8 presents a photographic comparison of the amorphous solid dispersion of curcumin using the NucleLoad™ processing method of Example 5 after storage for 3 weeks at 40° C./75% RH and the amorphous solid dispersion of curcumin using the conventional spray drying processing method of Example 8 after storage for 24 hours at 40° C./75% RH. This figure shows that the spray dried solid dispersion of Example 8 possesses inferior solid state stability compared to the NucleLoad™ solid dispersion of Example 5. The sample of Example 8 melted onto the sample tray into a glassy substance. It could not be isolated as a powder or processed further into a tablet or capsule. In stark contrast, the NucleLoad™ sample of Example 5 remained in a stable powder form after 3 weeks storage at 40° C./75% RH conditions, which are generally considered to be aggressive storage conditions for an amorphous solid dispersion.

While specific embodiments have been described above with reference to the disclosed embodiments and examples, such embodiments are only illustrative and do not limit the scope of the invention. Changes and modifications can be made in accordance with ordinary skill in the art without departing from the invention in its broader aspects as defined in the following claims.

All publications, patents, and patent documents are incorporated by reference herein, as though individually incorporated by reference. No limitations inconsistent with this disclosure are to be understood therefrom. The invention has been described with reference to various specific and preferred embodiments and techniques. However, it should be understood that many variations and modifications may be made while remaining within the spirit and scope of the invention.

What is claimed is:

1. A process for producing a solid dispersion comprising a material and a carrier, the steps of the process comprising:
(a) fluidizing a solid or liquid form of the carrier in a fluid bed to form a fluidized cloud of carrier particles; and
(b) spraying a liquid or gas form of the material onto the fluidized cloud of particles to form the solid dispersion.

2. The process of claim 1 where the material comprises a solution, emulsion or suspension.

3. The process of claim 1 where the material comprises a pharmaceutical ingredient, cosmetic, foodstuff, dietary supplement, flavoring, fragrance or specialty chemical.

4. The process of claim 1 where the carrier comprises a polysaccharide, a mineral or derivative comprising silicon, aluminum and/or magnesium, or a mixture thereof.

5. The process of claim 4 where the carrier comprises microcrystalline cellulose, alumina, clay, talc, kaolin, bentonite, hectorite, silicon dioxide, calcium silicate, colloidal magnesium-aluminum silicate, magnesium trisilicate, magnesium aluminometasilicate, aluminum hydroxide, magnesium hydroxide, magnesium oxide, or a mixture thereof.

6. The process of claim 5 where the silicon dioxide is fumed, colloidal, precipitated, milled, or a mixture thereof.

7. The process of claim 4 where the carrier has the following properties:
(a) a mean particle diameter of about 10-200 microns;
(b) a mean particle size of about 150 microns;
(c) a BET surface area of about 40-400 m²/g; and
(d) a pore volume of about 0.5-2.8 mL/g.

8. The process of claim 2 where the material comprises a pharmaceutical active ingredient and the carrier comprises a polysaccharide, a mineral or derivative comprising silicon, aluminum, and/or magnesium, or a mixture thereof.

9. The process of claim 8 where the solid dispersion comprises a void volume of less than about 70%.

10. The process of claim 8 where the active ingredient comprises a solution.

11. The process of claim 10 where the solution further comprises a pharmaceutical excipient.

12. The process of claim 10 where the solution comprises a solvent selected from the group consisting of water, acetone, ethyl acetate, dichloromethane, methanol ethanol, isopropanol, and a mixture thereof.

13. The process of claim 2 where the fluid bed contains a nozzle spraying configuration comprising a top spray, bottom spray or tangential/radial spray.

14. The process of claim 2 further comprising the step of compressing the solid dispersion into a tablet.

15. The process of claim 2 where the solid dispersion comprises a substantially amorphous form.

16. A process for producing a solid dispersion comprising a pharmaceutical active ingredient and a silica carrier, the steps of the process comprising:
(a) mixing a solid or gas form of the solid pharmaceutical active ingredient in a solvent to form a solution, suspension or emulsion;
(b) fluidizing a solid or liquid form of the silica carrier in a fluid bed to form a fluidized cloud of silica carrier particles; and
(c) spraying the solution, suspension or emulsion onto the fluidized cloud of silica carrier particles to form the solid dispersion.

17. The process of claim 16 where the silica carrier has the following properties:
(a) a mean particle diameter of about 10-200 microns;
(b) a mean particle size of about 150 microns;
(c) a BET surface area of about 40-400 m²/g; and
(d) a pore volume of about 0.5-2.8 mL/g.

18. The process of claim 16 where the solid dispersion comprises a void volume of less than about 70%.

19. The process of claim 16 where the solid dispersion comprises a substantially amorphous form.

20. A process for producing a solid dispersion comprising a pharmaceutical active ingredient and a silica carrier, the steps of the process comprising:
(a) mixing a solid or gas form of the pharmaceutical active ingredient in a solvent to form a solution, suspension or emulsion;
(b) fluidizing a solid or liquid form of the silica carrier in a fluid bed to form a fluidized cloud of silica carrier particles; and
(c) spraying the solution, suspension or emulsion onto the fluidized cloud of silica carrier particles to form the solid dispersion,
wherein,
the silica carrier has the following properties:
(a) a mean particle diameter of about 10-200 microns;
(b) a mean particle size of about 150 microns;
(c) a BET surface area of about 40-400 m²/g; and
(d) a pore volume of about 0.5-2.8 mL/g; and
the solid dispersion comprises a void volume of less than about 70%.
